(19) United States
(12) Patent Application Publication
(10) Pub. No.: US 2023/0310255 A1
(43) Pub. Date: Oct. 5, 2023

(54) PHANTOM THERAPY SYSTEM

(71) Applicant: Express Scripts Strategic Development, Inc., St. Louis, MO (US)

(72) Inventor: Moses Vaughan, Nutley, NJ (US)

(21) Appl. No.: 17/711,385

(22) Filed: Apr. 1, 2022

(51) Int. Cl. A61H 9/00; G16H 40/63; G16H 20/30

(52) U.S. Cl. CPC A61H 9/0078; G16H 40/63; G16H 20/30; A61H 2201/165; A61H 2201/5035; A61H 2201/5071; A61H 2201/5092; A61H 2201/5046; A61H 2201/5048; A61H 2201/5012

(57) ABSTRACT

Systems and methods herein describe a phantom therapy system. The phantom therapy system receives, via a network from a human machine interface pad, a set of pressure measurements, generates a set of pound-force per square inch (PSI) measurements based on the set of pressure measurements, transmits the set of PSI measurements to the set of pneumatic actuators coupled to a wearable fabric, and causes the set of pneumatic actuators to generate mechanical motion based on the set of PSI measurements.

### TECHNICAL FIELD

Embodiments of the present disclosure relate generally to virtual healthcare. More particularly, but not by way of limitation, the present disclosure addresses systems and methods for providing remote physical therapy over a computer network.

### BACKGROUND

Virtual health care can offer quality health care to those who may not have access to a physical medical treatment environment. Existing virtual health care treatment methods have been limited to video conferences and phone conversations.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a block diagram illustrating a phantom therapy system, according to example embodiments.

FIG. 2 is a block diagram of a phantom therapy controller, according to example embodiments.

FIG. 3 is an illustration of wearable fabric, according to example embodiments.

FIG. 4 is an example method for providing remote physical therapy using a phantom therapy controller, according to example embodiments.

FIG. 5 is a block diagram illustrating a software architecture, which can be installed on any one or more of the devices described herein.

FIG. 6 is a diagrammatic representation of the machine within which instructions (e.g., software, a program, an application, an applet, an app, or other executable code) for causing the machine to perform any one or more of the methodologies discussed herein may be executed.

### DETAILED DESCRIPTION

There is an existing need to expand the capacity of virtual health care to satisfy those that demand physical therapy treatment or other physical manipulations. Systems and methods herein address a phantom therapy system that provides physical therapy to remote clients and those demanding client centric data-driven care.

The phantom therapy system includes a human machine interface pad, a phantom therapy controller, and a wearable fabric. The phantom therapy system receives pressure measurements from a human machine interface pad. For example, the pressure measurements may be received by a health care provider providing tactile input on a human machine interface pad. Each of the pressure points and corresponding pressure measurements of the human machine interface pad may correspond to pneumatic actuators. The pneumatic actuators are physically coupled to a wearable fabric worn by a patient user. The phantom therapy system converts the pressure measurements of the human machine interface pad (typically measured in newtons) to pound-force per square inch (PSI) measurements. The phantom therapy system transmits the PSI measurements to the pneumatic actuators and causes the pneumatic actuators to generate mechanical motion based on the PSI measurements. The mechanical motion may replicate pressure and may also replicate 3D gestures (e.g., hand gestures of a health care provider).

The phantom therapy system may further include a camera that can capture and log the range of motion of a user wearing the wearable fabric. This benefits both the provider and patient by providing real-time percentage progress. This data can be stored in a database that is communicatively coupled to the phantom therapy system. The visual feedback also allows both the provider and patient with recommendations for further exercises that the patient can do to continue their physical therapy. The phantom therapy system may also include an audio unit that allows the patient and provider to verbally communicate during a remote physical therapy session. A patient can provide verbal feedback via the microphone to the provider to adjust the pressure settings of the wearable fabric. In some examples, the phantom therapy system further includes a display unit that allows for a patient to view pre-recorded therapy sessions. The pre-recorded therapy sessions may control the pneumatic actuators on the wearable fabric so as to replicate massage sensations or general hand movements. A patient may be able to select a pre-recorded session from the physical therapy system that best applies to them. Further details of the phantom therapy system are described below.

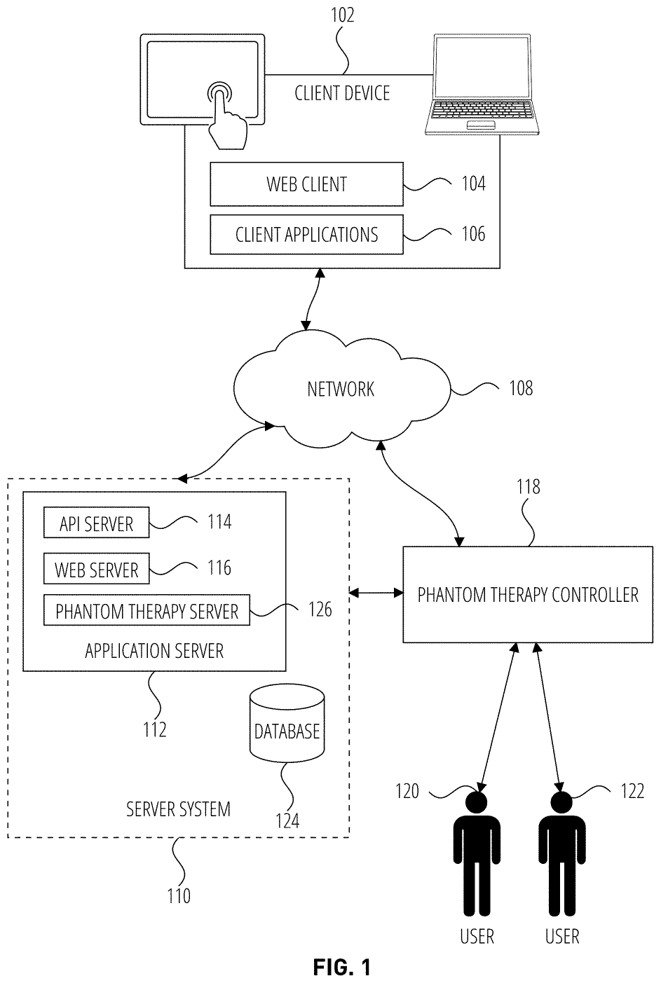

FIG. 1 is a block diagram illustrating a phantom therapy system, according to example embodiments, configured to provide physical therapy to one or more patients (eg., users) over a network. The system includes one or more client devices such as client device 102. The client device 102 comprises, but is not limited to, a mobile phone, desktop computer, laptop, portable digital assistants (PDA), smart phone, tablet, ultrabook, netbook, laptop, multi-processor system, microprocessor-based or programmable consumer electronic, game console, set-top box, computer in a vehicle, or any other communication device that a user may utilize to access the system 100. In some embodiments, the client device 102 comprises a display module (not shown) to display information (e.g., in the form of user interfaces). In further embodiments, the client device 102 comprises one or more of touch screens, accelerometers, gyroscopes, cameras, microphones, global positioning system (GPS) devices, and so forth. In some examples, the client device 102 is a human machine interface pad. The human machine interface pad may comprise sensors, hardware electronics and firmware that can detect multiple touch points with position and force. The client device 102 may be used by a health care provider who is providing remote physical therapy to one or more patients (e.g., users 120, 122).

One or more users may be a person, a machine, or other means of interacting with the client device 102. In example embodiments, the user may not be part of the phantom therapy system but may interact with the system via the client device 102. For instance, the user may provide input (e.g., touch screen input or alphanumeric input) to the client device 102 and the input may be communicated to other entities in the system (e.g., server system 110) via the network 108. In this instance, the other entities in the system, in response to receiving the input from the user, may communicate information to the client device 102 via the network 108 to be presented to the user. In this way, the user interacts with the various entities in the phantom therapy system using the client device 102.

The phantom therapy system further includes a network 108. One or more portions of network 108 may be an ad hoc network, an intranet, an extranet, a virtual private network (VPN), a local area network (LAN), a wireless LAN (WLAN), a wide area network (WAN), a wireless WAN (WWAN), a metropolitan area network (MAN), a portion of the Internet, a portion of the public switched telephone network (PSTN), a cellular telephone network, a wireless network, a WiFi network, a WiMax network, another type of network, or a combination of two or more such networks.

The client device 102 may access the various data and applications provided by other entities in the phantom therapy system via web client 104 (e.g., a browser) or one or more client applications 106. The client device 102 may include one or more client applications 106 (also referred to “apps”) such as, but not limited to, a web browser, a messaging application, electronic mail (email) application, a mapping or location application, and the like.

In some embodiments, one or more client applications 106 included in a given one of the client device 102, and configured to locally provide the user interface and at least some of the functionalities, with the client applications 106 configured to communicate with other entities in the system (e.g., server system 110, etc.), on an as-needed basis, for data processing capabilities not locally available (e.g., to access location information, to authenticate a user, etc.). Conversely, one or more client applications 106 may not be included in the client device 102, and then the client device 102 may use its web browser to access the one or more applications hosted on other entities in the phantom therapy system (e.g., server system 110, etc.).

A server system 110 provides server-side functionality via the network 108 (e.g., the Internet or wide area network (WAN)) to: one or more client devices 102. The server system 110 includes an application program interface (API) 114, a web server 116, and a phantom therapy server 126, that may be communicatively coupled with one or more databases 124. The one or more databases 124 may be storage devices that store data related to users of the server system 110, applications associated with the server system 110, cloud services, phantom therapy data, and so forth. The one or more databases 124 may further store information related to client device 102, client applications 106, users 120 and 122, and so forth. In one example, the one or more databases 124 may be cloud-based storage.

The server system 110 may be a cloud computing environment, according to some example embodiments. The server system 110 and any servers associated with the server system 110, may be associated with a cloud-based application, in one example embodiment.

The server system 110 includes a phantom therapy server 126. The phantom therapy server 126 may be associated with a cloud-based application. The phantom therapy server 126 may be used to distribute physical therapy sessions to one or more patients (e.g., user 120, 122) simultaneously or on an individualized basis. In some examples the distributed physical therapy sessions may be pre-recorded therapy sessions.

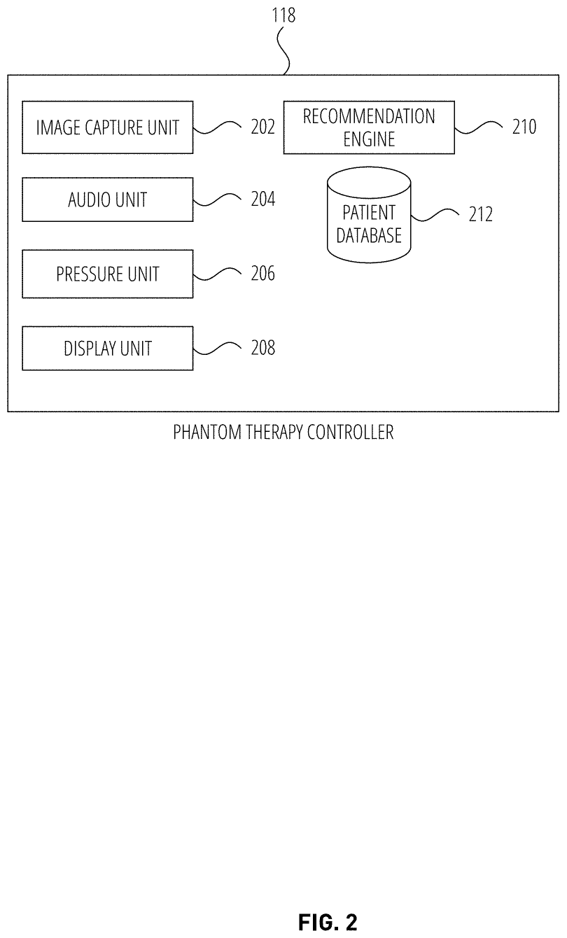

The phantom therapy system may further include a phantom therapy controller 118. The phantom therapy controller 118 is a hardware device that receives pressure information from the client device 102 (e.g., a human machine interface pad) and communicates the pressure information to a wearable fabric. The wearable fabric is a fabric that is lined with arrays of tactile actuators. The actuators may be pneumatic actuators attached to the fabric. The phantom therapy controller 118 may trigger the pneumatic actuators of the wearable fabric to simulate the pressure and/or gestures received as input on the client device 102. Further details of the phantom therapy controller 118 are found in FIGS. 2-4.

FIG. 2 is a block diagram of a phantom therapy controller 118, according to example embodiments. The phantom therapy controller 118 is shown to include an image capture unit 202, an audio unit 204, a pressure unit 206, a display unit 208, a recommendation engine 210, and a patient database 212.

The image capture unit 202 includes one or more cameras. The image capture unit 202 may be used to capture a range of motion of a patient (e.g., user 120 or user 122) during a remote physical therapy session. The image capture unit 202 may further include one or more processors configured to perform various image processing algorithms including object tracking, face detection, pose detection, and the like. It is to be understood that the image capture unit 202 may employ any suitable digital image processing techniques. In some examples, a patient may be able to control the phantom therapy controller 118 with hand gestures (e.g., a thumbs down gesture may cause the phantom therapy controller 118 to decrease pressure of the pneumatic actuators while a thumbs up gesture may cause the phantom therapy controller 118 to increase pressure of the pneumatic actuators).

The audio unit 204 includes one or more speakers and microphones. The audio unit 204 may be used to allow the patient (e.g., user 120, 122) and a provider (e.g., user of client device 102) to verbally communicate during a remote physical therapy session. In some examples, a patient may be able to provide the phantom therapy controller 118 with audio commands to control functionality of the phantom therapy controller 118 (e.g., a user 120 may say “Stop” and the phantom therapy controller 118 will cause the pneumatic actuators on the wearable fabric to release all pressure).

The pressure unit 206 is configured to convert the pressure information received by the client device 102 into pressure measurements that can be transmitted to the pneumatic actuators of the wearable fabric. In some examples, the pressure information received by the client device 102 is measured in newtons (N). The pressure information may need to be converted into a form of measurement that is understood by the pneumatic system (e.g., the pneumatic actuators and a corresponding air line connected to a tank of air or pressurized gas). In some examples, the pneumatic system may require receiving pressure data in terms of pound-force per square inch (PSI). The pressure unit 206 may use an application programming interface (API) to convert the pressure information received by the client device 102 (e.g., measured in newtons) to pressure information consumable by the pneumatic system (e.g., measured in PSI). While the discussion above describes converting pressure information from newtons to PSI, it is to be understood, that the pressure information may be measured in any suitable unit of measure including Newton, PSI, Pascal, and the like.

The display unit 208 includes a graphical user interface (GUI). The GUI may allow a patient to view pre-recorded therapy sessions. The graphical user interface may include user interface elements (buttons, checkboxes, scroll bars, etc.). A patient may select a pre-recorded therapy session from the GUI using one or more user interface elements. Selection of the one or more user interface elements may cause the pneumatic actuators in the wearable fabric to replicate massage sensations or general hand movements. The display unit 208 may further be controlled by voice commands or hand gestures by the patient user.

The recommendation engine 210 uses data from the patient database 212 to provide recommended exercises for a patient. Aspects of the recommendation engine 210 may exist on the phantom therapy controller 118 and other aspects may exist on the server system 110. In some examples, the recommendation engine 210 operates exclusively on the phantom therapy controller 118.

The patient database 212 may include progress data of the patient (e.g., captured by the image capture unit 202 or audio unit 204). The patient database 212 may further include data associated with others who fall in a similar demographic category of the patients to facilitate comparing the patient's progress with others in his or her demographic. The recommendation engine 210 may use the data in the patient database 212 to determine if the patient user is ahead of schedule in their physical therapy progress or behind schedule. Based on the determination, the recommendation engine 210 may provide recommended pre-recorded therapy sessions as discussed above, to the patient. The recommended therapy sessions may be ranked higher than other therapy sessions that are available to the user. For example, the recommended therapy sessions may be presented in a first (main) portion of the GUI of the phantom therapy controller 118, while other therapy sessions may be presented a second portion of the GUI.

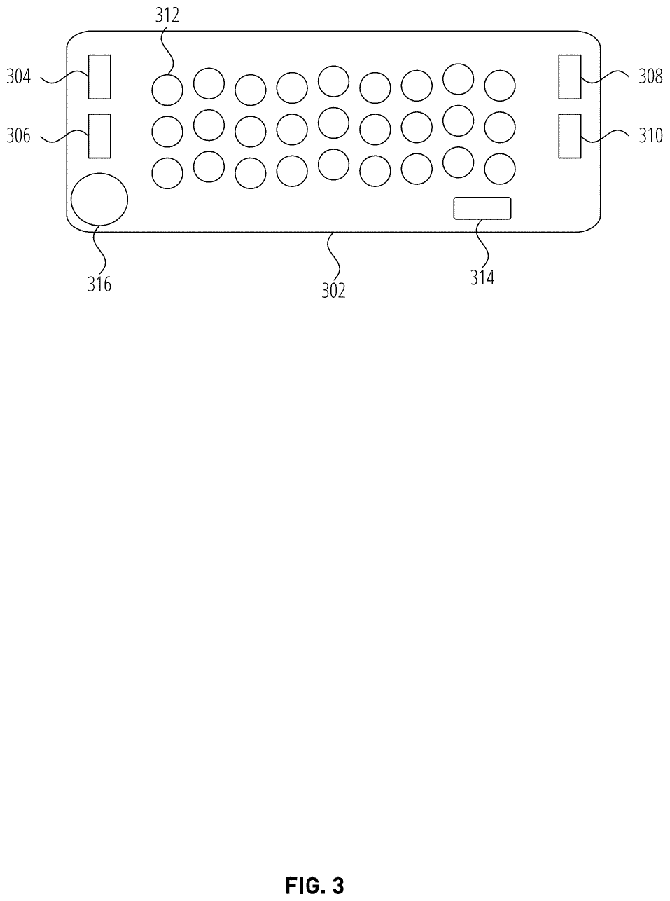

FIG. 3 is an illustration of wearable fabric 302, according to example embodiments. The wearable fabric 302 is shown to include a series of pneumatic actuators 312, an air line 316 that is physically connected to a tank of air or pressurized gas (not pictured), a feedback sensor 314, and adjustable straps 304, 306, 308, and 310. The series of pneumatic actuators 312 may be placed in separate pockets (not pictured) within the fabric 302. The adjustable straps may be, for example, VELCRO straps. It is to be understood that the adjustable straps 304, 306, 308, 310 may be any suitable material. The wearable fabric may be wrapped around a body part such as an arm or leg and multiple wearable fabrics may be used to obtain more coverage or work on multiple parts of the body. Although the wearable fabric is shown as a strip of fabric, the wearable fabric may replicate any article of clothing. In some examples, the wearable fabric may be in the shape of a shirt, sleeve, or glove and a combination of these fabrics may be used to expand the network of physical sensations a user can experience.

When a health care provider applies pressure to a human machine interface pad (e.g., client device 102), the phantom therapy controller 118 receives the applied pressure and instructs the pneumatic actuators 312 in the wearable fabric 302 to receive the applied pressure from the tank of air or pressurized gas via the air line 316. The pneumatic actuators 312 receive the pressure from the tank of air or pressurized gas and convert the pressure into mechanical motion. This approach enables a health care provider to deliver fine grain pressure over a network, which can be customized to a user's specific needs. It is to be understood that the pneumatic actuators 312 may be implemented in any suitable method that involves converting energy into mechanical motion.

The feedback sensor 314 may be a button that allows the wearer of the wearable fabric to adjust the pressure of the pneumatic actuators 312. A user (120, 122) may be able to increase or decrease the overall pressure of the pneumatic actuators 312. The phantom therapy controller 118 may be able to send the feedback information collected by the feedback sensor 314 back to the client device 102 such that a healthcare provider can adjust the corresponding pressure settings.

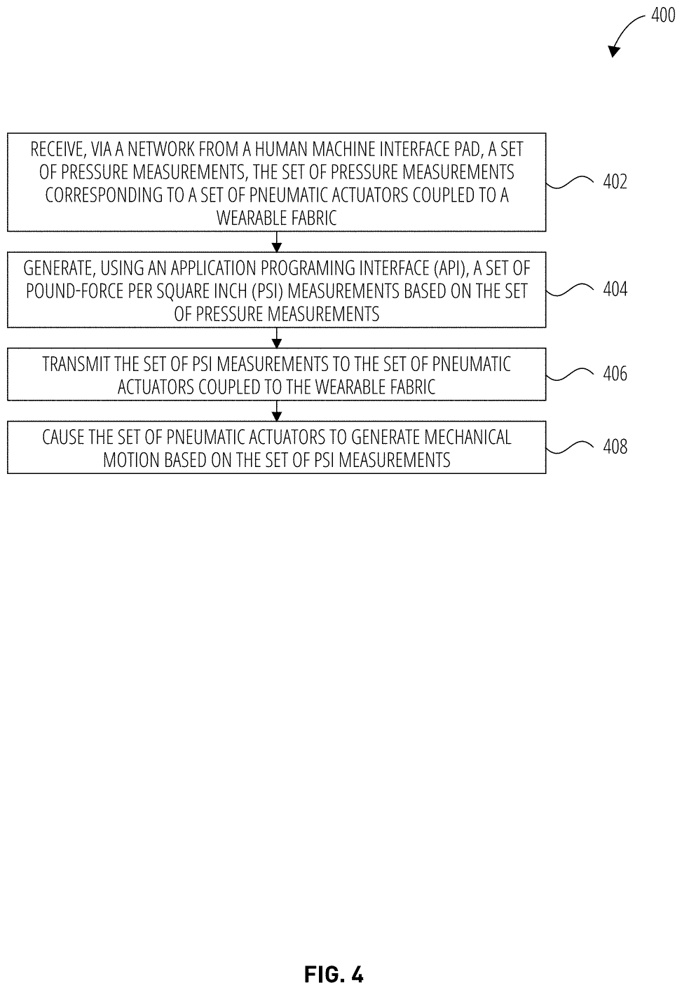

FIG. 4 is an example method 400 for providing remote physical therapy using a phantom therapy controller 118, according to example embodiments. Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems. In some examples, one or more processors of the phantom therapy server 126, the phantom therapy controller 118 or a combination thereof perform the operations in method 400.

At operation 402, the phantom therapy controller 118 receives a set of pressure measurements. The set of pressure measurements are received via a network from a human machine interface pad (e.g., client device 102. The set of pressure measurements correspond to a set of pneumatic actuators coupled to a wearable fabric (e.g., wearable fabric 302). Although the method 400 describes controlling a single wearable fabric, it is to be understood that multiple wearable fabrics may be controlled using the operations described in method 400.

At operation 404, the phantom therapy controller 118 generates, using an application programing interface (API), a set of pound-force per square inch (PSI) measurements based on the set of pressure measurements. For example, the phantom therapy controller 118 may convert the pressure measurements received by the human machine interface pad (measured in newtons) to PSI measurements. Operations 402 and 404 may be performed by the pressure unit 206.

At operation 406, the phantom therapy controller 118 transmits the set of PSI measurements to the set of pneumatic actuators 312 coupled to the wearable fabric 302. At operation 408, the phantom therapy controller 118 causes the set of pneumatic actuators 312 to generate mechanical motion based on the set of PSI measurements.

In some embodiments, the phantom therapy controller 118 captures a set of images (which for purposes of this disclosure may also include video) using an image capture device. The set of images comprise a range of motion of a human body part, wherein the wearable fabric is physically attached to the human body part. The images may be captured by the image capture unit 202. Image data and analysis corresponding to the range of motion may be stored in the patient database 212. The analysis may include further data points regarding the motion (e.g., degree of motion, comparison to a threshold motion range). The analysis may also be referred to as motion data.

In some embodiments, the phantom therapy controller 118 records, using a microphone, audio of a user 120 or user 122 wearing the wearable fabric 302. The audio may be recorded using the audio unit 204. In some examples, the recorded audio may be stored in the patient database 212.

In some embodiments, the phantom therapy controller 118 causes display on a graphical user interface (GUI) of the phantom therapy controller 118, a set of pre-recorded therapy programs. The display may be the display unit 208. The phantom therapy controller 118 may receive a selection from the GUI of a user interface element by a user. The selection may include one or more pre-recorded therapy programs from the set of pre-recorded therapy programs. The set of pre-recorded therapy programs may be prioritized and ranked according to the recommendation engine 210. The set of pre-recorded therapy programs may be displayed based on user progress of the user. For examples, if the user is responding well to the therapy (e.g., the user is progressing faster than other users of the same demographic receiving similar type of therapy), then the set of pre-recorded therapy programs include more rigorous therapy exercises. If the user is behind schedule and is not progressing as expected (e.g., the user is progressing slower than other users of the same demographic receiving similar type of therapy), then the set of pre-recorded therapy programs include less rigorous therapy exercises. In some examples, the user progress of the user is displayed on the GUI as a graphical element (e.g., a chart, bar graph, or other suitable illustration). The graphical element includes a comparison of the user progress of the user in relation to a second set of user progress data. The second set of user progress data may include data of demographically similar user's and their corresponding user progress to similar forms of therapy.

In some embodiments, the phantom therapy controller 118 receives, from one or more sensors coupled to the wearable fabric, feedback from a user wearing the wearable fabric. The feedback may be received from the feedback sensor 314. In response to receiving the feedback from the feedback sensor, the phantom therapy controller 118 may generate an adjusted set of PSI measurements (e.g., decreased pressure measurements or increased pressure measurements than the current pressure settings). The phantom therapy controller 118 may transmit the adjusted set of PSI measurements to the set of pneumatic actuators coupled to the wearable fabric.

In some embodiments, the phantom therapy controller 118 receives pressure data from multiple wearable fabrics worn by the user. For example, the phantom therapy controller 118 receives a second set of pressure measurements from the human machine interface pad and generates a second set of PSI measurements based on the second set of pressure measurements. The phantom therapy controller 118 transmits the second set of PSI measurements to a second set of pneumatic actuators coupled to the second wearable fabric. The phantom therapy controller 118 causes the first set of pneumatic actuators to generate mechanical motion based on the first set of PSI measurements and the second set of PSI measurements. The phantom therapy controller 118 may further cause the second set of pneumatic actuators to generate mechanical motion based on the second set of PSI measurements. In some examples, the second wearable fabric is worn on a different body part of the user than the first wearable fabric. In other examples, the second wearable fabric may be located proximate to the first wearable fabric.

Software Architecture

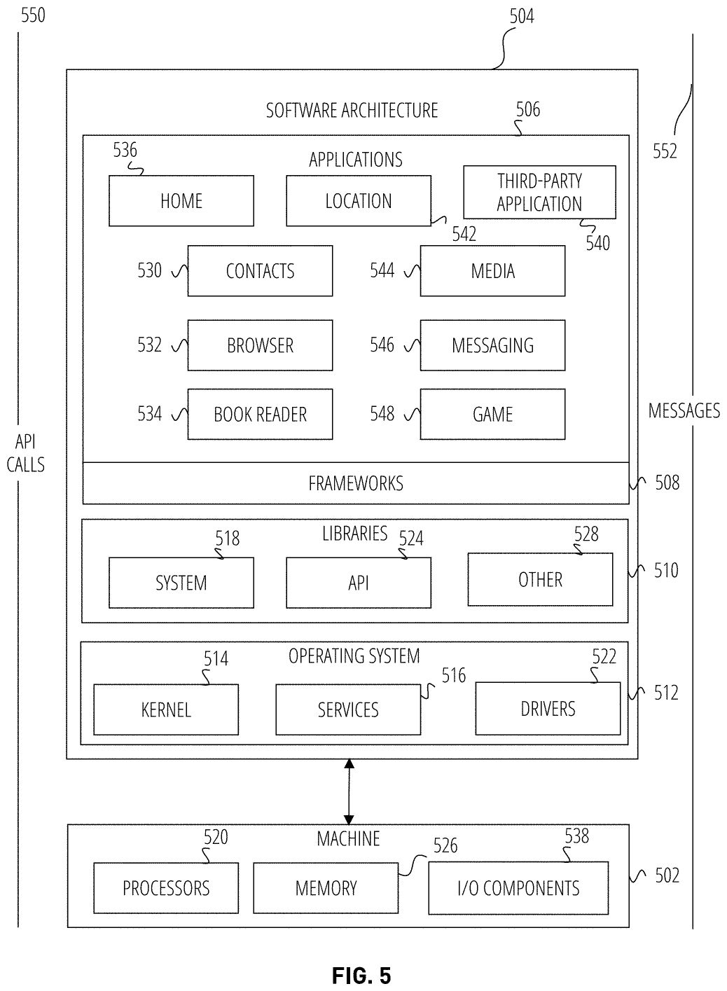

FIG. 5 is a block diagram illustrating a software architecture 504, which can be installed on any one or more of the devices described herein. The software architecture 504 is supported by hardware such as a machine 502 that includes processors 520, memory 526, and I/O components 538. In this example, the software architecture 504 can be conceptualized as a stack of layers, where each layer provides a particular functionality. The software architecture 504 includes layers such as an operating system 512 libraries 510, frameworks 508, and applications 506. Operationally, the applications 506 invoke API calls 550 through the software stack and receive messages 552 in response to the API calls 550.

The operating system 512 manages hardware resources and provides common services. The operating system 512 includes, for example, a kernel 514, services 516, and drivers 522. The kernel 514 acts as an abstraction layer between the hardware and the other software layers. For example, the kernel 514 provides memory management, processor management (e.g., scheduling), component management, networking, and security settings, among other functionality. The services 516 can provide other common services for the other software layers. The drivers 522 are responsible for controlling or interfacing with the underlying hardware. For instance, the drivers 522 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 510 provide a common low-level infrastructure used by the applications 506. The libraries 510 can include system libraries 518 (e.g., C standard library) that provide functions such as memory allocation functions, string manipulation functions, mathematic functions, and the like. In addition, the libraries 510 can include API libraries 524 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 510 can also include a wide variety of other libraries 528 to provide many other APIs to the applications 506.

The frameworks 508 provide a common high-level infrastructure that is used by the applications 506. For example, the frameworks 508 provide various graphical user interface (GUI) functions, high-level resource management, and high-level location services. The frameworks 508 can provide a broad spectrum of other APIs that can be used by the applications 506, some of which may be specific to a particular operating system or platform.

In an example, the applications 506 may include a home application 536, a contacts application 530, a browser application 532, a book reader application 534, a location application 542, a media application 544, a messaging application 546, a game application 548, and a broad assortment of other applications such as a third-party application 540 The applications 506 programs that execute functions defined in the programs. Various programming languages can be employed to create one or more of the applications 506 structured in a variety of manners, such as object-oriented programming languages (e.g., Objective-C, Java, or C++) or procedural programming languages (e.g., C or assembly language). In a specific example, the third-party application 540 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 540 can invoke the API calls 550 provided by the operating system 512 to facilitate functionality described herein.

Machine Architecture

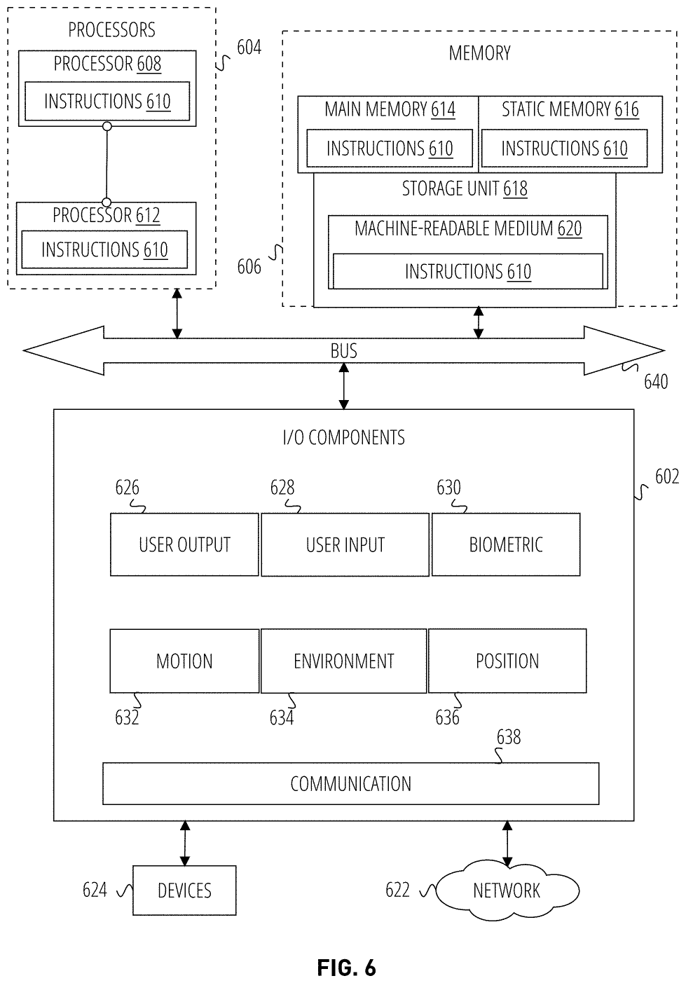

FIG. 6 is a diagrammatic representation of the machine within which instructions 610 (e.g., software, a program, an application, an applet, an app, or other executable code) for causing the machine to perform any one or more of the methodologies discussed herein may be executed. For example, the instructions 610 may cause the machine to execute any one or more of the methods described herein. The instructions 610 transform the general, non-programmed machine into a particular machine programmed to carry out the described and illustrated functions in the manner described. The machine may operate as a standalone device or may be coupled (e.g., networked) to other machines. In a networked deployment, the machine may operate in the capacity of a server machine or a client machine in a server-client network environment, or as a peer machine in a peer-to-peer (or distributed) network environment. The machine may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 610, sequentially or otherwise, that specify actions to be taken by the machine. Further, while only a single machine is illustrated, the term “machine” shall also be taken to include a collection of machines that individually or jointly execute the instructions 610 to perform any one or more of the methodologies discussed herein. The machine, for example, may comprise the client device 102, the phantom therapy controller 118, or any one of a number of server devices in a phantom therapy system. In some examples, the machine may also comprise both client and server systems, with certain operations of a particular method or algorithm being performed on the server-side and with certain operations of the particular method or algorithm being performed on the client-side.

The machine may include processors 604, memory 606, and input/output I/O components 638, which may be configured to communicate with each other via a bus 640. In an example, the processors 604 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 608 and a processor 608 that execute the instructions 610. The term “processor” is intended to include multi-core processors that may comprise two or more independent processors (sometimes referred to as “cores”) that may execute instructions contemporaneously. Although FIG. 6 shows multiple processors 604, the machine may include a single processor with a single-core, a single processor with multiple cores (e.g., a multi-core processor), multiple processors with a single core, multiple processors with multiples cores, or any combination thereof.

The memory 606 includes a main memory 614, a static memory 616 and a storage unit 618 both accessible to the processors 604 via the bus 640. The main memory 614, a static memory 616 and storage unit 618 both store the instructions 610 embodying any one or more of the methodologies or functions described herein. The instructions 610 may also reside, completely or partially, within the main memory 614, within the static memory 616, within machine-readable medium 620 within the storage unit 618, within at least one of the processors 604 (e.g., within the Processor's cache memory), or any suitable combination thereof, during execution thereof by the machine.

The I/O components 602 may include a wide variety of components to receive input, provide output, produce output, transmit information, exchange information, capture measurements, and so on. The specific I/O components 602 that are included in a particular machine will depend on the type of machine. For example, portable machines such as mobile phones may include a touch input device or other such input mechanisms, while a headless server machine will likely not include such a touch input device. It will be appreciated that the I/O components 602 may include many other components that are not shown in FIG. 6. In various examples, the I/O components 602 may include user output components 626 and user input components 628. The user output components 626 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 628 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 602 may include biometric components 630, motion components 632, environmental components 634, or position components 636, among a wide array of other components. For example, the biometric components 630 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 632 include acceleration sensor components (e.g., accelerometer), gravitation sensor components, rotation sensor components (e.g., gyroscope).

The environmental components 634 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

The position components 636 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 602 further include communication components 638 operable to couple the machine to a network 622 or devices 624 via respective coupling or connections. For example, the communication components 638 may include a network interface component or another suitable device to interface with the network 622. In further examples, the communication components 638 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 624 may be another machine or any of a wide variety of peripheral devices (e.g., a peripheral device coupled via a USB).

Moreover, the communication components 638 may detect identifiers or include components operable to detect identifiers. For example, the communication components 638 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 638, such as location via Internet Protocol (IP) geolocation, location via Wi-Fi® signal triangulation, location via detecting an NFC beacon signal that may indicate a particular location, and so forth.

The various memories (e.g., main memory 614, static memory 616, and memory of the processors 604) and storage unit 618 may store one or more sets of instructions and data structures (e.g., software) embodying or used by any one or more of the methodologies or functions described herein. These instructions (e.g., the instructions 610)), when executed by processors 604, cause various operations to implement the disclosed examples.

The instructions 610 may be transmitted or received over the network 622, using a transmission medium, via a network interface device (e.g., a network interface component included in the communication components 638) and using any one of several well-known transfer protocols (e.g., hypertext transfer protocol (HTTP)). Similarly, the instructions 610 may be transmitted or received using a transmission medium via a coupling (e.g., a peer-to-peer coupling) to the devices 624.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A method comprising:
receiving, via a network from a human machine interface pad, a first set of pressure measurements, the first set of pressure measurements corresponding to a first set of pneumatic actuators coupled to a first wearable fabric;
generating, using an application programing interface (API), a first set of pound-force per square inch (PSI) measurements based on the first set of pressure measurements;
transmitting the first set of PSI measurements to the first set of pneumatic actuators coupled to the wearable fabric; and
causing the first set of pneumatic actuators to generate mechanical motion based on the first set of PSI measurements.

2. The method of claim 1, wherein the first set of pressure measurements is measured in newtons.

3. The method of claim 1, further comprising:
capturing, a set of images using an image capture device, the set of images comprising a range of motion of a human body part, wherein the first wearable fabric is physically attached to the human body part.

4. The method of claim 1, further comprising:
recording, using a microphone, audio of a user wearing the first wearable fabric.

5. The method of claim 1, further comprising:
storing motion data in a database, wherein the motion data is associated with the first set of pneumatic actuators.

6. The method of claim 1, further comprising:
causing display on a graphical user interface of a computing device, a set of pre-recorded therapy programs.

7. The method of claim 6, further comprising:
receiving a selection of a user interface element by a user, the selection comprising one or more pre-recorded therapy programs from the set of pre-recorded therapy programs.

8. The method of claim 6, wherein the set of pre-recorded therapy programs are displayed based on user progress associated with a user wearing the first wearable fabric.

9. The method of claim 8, further comprising:
causing display of the user progress on the graphical user interface of the computing device, the user progress displayed as a graphical element, the graphical element comprising a comparison of the user progress in relation to a second set of user progress data.

10. The method of claim 1, further comprising:
receiving from the human machine interface pad, a second set of pressure measurements, the second set of pressure measurements corresponding to a second set of pneumatic actuators coupled to a second wearable fabric;
generating a second set of pound-force per square inch (PSI) measurements based on the second set of pressure measurements; and
causing the first set of pneumatic actuators to generate mechanical motion based on the first set of PSI measurements and the second set of PSI measurements.

11. The method of claim 1, further comprising:
receiving, from one or more sensors coupled to the first wearable fabric, feedback from a user wearing the first wearable fabric; and
in response to receiving the feedback, generating a first adjusted set of PSI measurements; and
transmitting the first adjusted set of PSI measurements to the first set of pneumatic actuators coupled to the first wearable fabric.

12. A system comprising:
a processor; and
a memory storing instructions that, when executed by the processor, configure the system to:
receive, via a network from a human machine interface pad, a first set of pressure measurements, the first set of pressure measurements corresponding to a first set of pneumatic actuators coupled to a first wearable fabric;
generate, using an application programing interface (API), a first set of pound-force per square inch (PSI) measurements based on the first set of pressure measurements;
transmit the first set of PSI measurements to the first set of pneumatic actuators coupled to the first wearable fabric; and
cause the first set of pneumatic actuators to generate mechanical motion based on the first set of PSI measurements.

13. The system of claim 12, wherein the first set of pressure measurements is measured in newtons.

14. The system of claim 12, wherein the instructions further configure the system to:
capture, a set of images using an image capture device, the set of images comprising a range of motion of a human body part, wherein the first wearable fabric is physically attached to the human body part.

15. The system of claim 12, wherein the instructions further configure the system to:
record, using a microphone, audio of a user wearing the first wearable fabric.

16. The system of claim 12, wherein the instructions further configure the system to:
store motion data in a database, wherein the motion data is associated with the first set of pneumatic actuators.

17. The system of claim 12, wherein the instructions further configure the system to:
cause display on a graphical user interface of a computing device, a set of pre-recorded therapy programs.

18. The system of claim 17, wherein the instructions further configure the system to:
receive a selection of a user interface element by a user, the selection comprising one or more pre-recorded therapy programs from the set of pre-recorded therapy programs.

19. The system of claim 12, wherein the instructions further configure the system to:
receive, from one or more sensors coupled to the first wearable fabric, feedback from a user wearing the first wearable fabric; and
in response to receiving the feedback, generate a first adjusted set of PSI measurements; and
transmit the first adjusted set of PSI measurements to the first set of pneumatic actuators coupled to the first wearable fabric.

20. A non-transitory computer-readable storage medium, the computer-readable storage medium including instructions that when executed by a computer, cause the computer to:
receive, via a network from a human machine interface pad, a first set of pressure measurements, the first set of pressure measurements corresponding to a first set of pneumatic actuators coupled to a first wearable fabric;
generate, using an application programing interface (API), a first set of pound-force per square inch (PSI) measurements based on the first set of pressure measurements;
transmit the first set of PSI measurements to the first set of pneumatic actuators coupled to the first wearable fabric; and
cause the first set of pneumatic actuators to generate mechanical motion based on the first set of PSI measurements.
